SEQUENCE LISTING (8 sequences)

SEQ ID NO: 1 - protein, 15 aa, Artificial Sequence
  DBY peptide
NAGFNSNRANSSRSS

SEQ ID NO: 2 - protein, 9 aa, Artificial Sequence
  UTY peptide
WMHHNMDLI

SEQ ID NO: 3 - DNA, 19 bp, artificial sequence
  Foxp3 1 initiator
ggcccttctccaggacaga

SEQ ID NO: 4 - DNA, 20 bp, artificial sequence
  Foxp3 2 initiator
gctgatcatggctgggttgt

SEQ ID NO: 5 - DNA, 28 bp, artificial sequence
  Foxp3 3 initiator
acttcatgcatcagtctccactgtggat

SEQ ID NO: 6 - DNA, 20 bp, artificial sequence
  mPO initiator
ctccaagcagatgcagcaga

SEQ ID NO: 7 - DNA, 20 bp, artificial sequence
  mPO 2 initiator
atagccttgcgcatcatggt

SEQ ID NO: 8 - DNA, 22 bp, artificial sequence
  mPO 3 initiator
ccgtggtgctgatgggcaagaa

(19) United States
(12) Patent Application Publication
(10) Pub. No.: US 2009/0220468 A1
(43) Pub. Date: Sep. 3, 2009

(54) Specific CD4+CD25+ Regulatory T Cells for Haematopoietic Cell Transplantation and Immune Tolerance

(75) Inventors: David Alexandre Gross, Vincennes (FR); Jean Davoust, Saint Germain en Laye (FR)

Correspondence Address: HESLIN ROTHENBERG FARLEY & MESITI PC, 5 COLUMBIA CIRCLE, ALBANY, NY, 12203, US

(73) Assignees: GENETHON, Evry (FR); CENTRE NATIONAL DE LA RECHERCHE SCIENTIFIQUE, Paris (FR)

(21) Appl. No.: 12/281,782

(22) PCT Filed: Mar. 9, 2007
(86) PCT No.: PCT/EP2007/052222

§ 371 Date: Feb. 9, 2009

(30) Foreign Application Priority Data
Mar. 10, 2006 (FR) 0650828

(51) Int. Cl. A61K 35/12

(52) U.S. Cl. 424/93.21; 424/93.7

(57) ABSTRACT

A pharmaceutical composition comprising CD4+ CD25+ regulatory T cells specific for at least one minor histocompatibility antigen, and stem cells, advantageously haematopoietic, carrying at least the antigen can be used as a medicament for increasing the immune tolerance of a histocompatible host.

The invention relates to CD4+CD25+ regulatory T cells specific for a minor antigen, and more precisely their combination with haematopoietic stem cells in a pharmaceutical composition, and their use as a medicament to increase the immune tolerance of a histocompatible host, including to transgenes.

Gene replacement therapy has aroused considerable interest for treating various metabolic diseases and genetic disorders, such as muscular dystrophy, or haemophilia. However, it is very important to avoid deleterious immune responses. Tremendous efforts have been made to improve the gene delivery procedures, seeking the least immunogenic vectors, tissue-specific promoters, and appropriate injection methods and doses (1, 2). However, these approaches are faced with the risk of creating a status of immune ignorance, in which a secondary inflammation or signals of neighbouring pathogens can induce an immune rejection of the gene therapy products (3). The induction of peripheral tolerance using immuno-suppressive co-treatments or the transient blocking of co-stimulation passages, initially developed for transplantation or autoimmunity applications, have been successfully transposed to gene therapy (4, 5), but these procedures are accompanied by a pronounced toxicity, increasing the risks of cardiovascular disease, opportunistic infections and malignant tumours in humans. Specific tolerance approaches to transgenes are necessary to block the initial rejection and to induce a long-term tolerance towards transgenes, without affecting the general immune functions of the patients. These requirements are satisfied by expressing the transgenes of interest in the haematopoietic system before the gene therapy operations.

Mixed haematopoietic chimerism, with the robust donor-specific tolerance associated with it, can be used for organ transplantation (6) and the treatment of autoimmune diseases (7). Mixed haematopoietic chimerism was recently used to induce a tolerance towards specific proteins by transferring genes to autologous haematopoietic stem cells (HSC) for gene therapy (8). However, its stable induction demands a long-term immuno-depression and myeloablative conditioning to prevent the rejection of the transplant and to create a space in the micro environment of the host marrow. Thus, a bone marrow transplantation requires a partial myeloablation combined with an immuno-depression, to achieve the necessary mixed haematopoietic chimerism.

High doses of bone marrow cells (BM) can overcome the limited accessibility of the available niches (9, 10). However, even in the case of optimal BM transplants compatible for HLA histocompatibility molecules, minor histocompatibility antigens (AgMH) can cause an immune rejection (11, 12), requiring an intensive immuno-depression. Foreign transgenes expressed in the haematopoietic system cells (HSC) constitute minor histocompatibility antigens which can compromise the engraftment of the HSC transplant.

Among the sub-groups of regulatory T lymphocytes, the CD4+CD25+ T cells (Tregs), governed by the forkhead transcription factor Foxp3, are particularly promising for inducing tolerance in transplantation (12, 13), because they can forestall the disease of the graft against the host (14, 15) and inhibit the rejection of BM allograft in sublethally irradiated recipients (16). Although the recognition of antigens (17) is suspected of being involved in immune regulation by Tregs, their in vivo action mechanism remains to be clarified. The modalities of the use of this cell population for potential therapeutic applications also still need to be determined.

The technical problem that the present invention proposes to solve is therefore to find an alternative treatment to the conventional myeloablative immune-depressive treatment, which is considered to be lengthy, cumbersome and potentially dangerous with regard to infectious and tumoral risks which it usually generates in the recipient.

In the context of the present invention, the Applicant has demonstrated the antigen-specific immuno-depressive potential of a particular class of regulatory T cells, which constitutes an alternative for the conditioning of the recipient for a transplantation for example. Thus, a simple conditioning with this cell population serves to increase the long-term immune tolerance of a histocompatible host.

According to a first aspect, the invention therefore relates to a pharmaceutical composition comprising:
CD4+CD25+ regulatory T cells specific for at least one minor histocompatibility antigen; and
stem cells, advantageously haematopoietic, carrying at least the said antigen.

Firstly, it must be pointed out that in the rest of the description, the invention relates to the introduction of cells or tissues into histocompatible hosts. This implies that the graft and the host have an identical major histocompatibility system (HLA) and are therefore haploidentical. This is referred to as an allograft.

In this case, the reaction or even the rejection of the transplant by the host is associated with the presence, in the grafted HSC, of non-HLA antigens, called minor histocompatibility antigens (AgMH), specific for the transplantation (and therefore the donor) and divergent or even absent in the host (recipient). These minor antigens, originating both from intracellular proteins and surface proteins, are presented in the context of molecules of the HLA system.

By way of illustration, the transfer of male cells or tissues to a female yet histocompatible host is suspected of triggering an immune reaction, due to the presence of male minor antigens on the cells or the tissue. These male antigens are coded for the Y chromosome and are preferably the proteins DBY, UTY or SMCY. These particular antigens have the advantage of being correctly expressed (effective) and well preserved (little polymorphism) in the various individuals, which makes them antigens called universal antigens.

The active principle of a pharmaceutical composition according to the invention resides in the CD4+CD25+ regulatory T cells specific of at least one AgMH antigen as defined above. In other words, these regulatory T cells are capable of specifically recognizing this AgMH by the intermediary of their T receptor or of any other specific recognition mechanism.

The regulatory T cells (Treg) correspond to a sub-population of T lymphocytes—blood cells involved in the correct functioning of the immune system—capable of peripherally neutralizing the destructive action of the autoreactive T lymphocytes. These regulatory cells are involved in preventing the initiation of autoreactive immune reactions, the basis of autoimmune pathologies. The CD4+CD25+ regulatory T cells are easily identified, isolated and purified thanks to the presence of the markers CD4 and CD2. They are also characterized by the fact that they specifically express the transcription factor Foxp3, recently identified in this cell population.

In the context of the present invention, the expression “specific foreign antigen” means that the T cells express TCR surface recipients capable of recognizing and interacting with at least one epitope of the AgMH antigen. Similarly, it is stated that the T cells are directed against the said antigens.

These Treg cells are capable of recognizing at least one antigen. This means that they may be monospecific, recognizing one epitope of an antigen. Alternatively, they may be directed against a plurality of donor AgMH antigens. It may also be considered to direct these cells against several distinct epitopes of the same antigen.

Advantageously, the T cells used in the context of the invention are specific for a single AgMH antigen, thereby proving the power of the technical solution proposed, which is capable of conferring a broad immune tolerance.

In the particular case of the recognition of DBY, such a cell population has been produced by mice carrying a transgenic T cell recipient, specific of the peptide DBY (NAGFNSNRANSSRSS) (SEQ ID 1), complexed with IA^{b}. Such mice are known by the name of Marylin mice (18).

Such monospecific T cells can now be cultivated and multiplied in vitro thanks to the protocol described by Tarbel et aL (19). Furthermore, it is also possible to generate this type of cell de novo in humans (20).

Ultimately, a pharmaceutical composition according to the invention will contain the factors necessary for the de novo generation in the host of T cells as defined above.

In an advantageous embodiment, the T cells as defined above are autologous, that is they originate from the donor who, in this particular case, is also the recipient.

In combination with this first cell population (Tregs), the pharmaceutical composition further comprises a second type of cell, that is stem cells. For ethical reasons, the present invention does not relate to stem cells directly issuing from human embryos.

These cells carry and express at least the antigen that differs in the recipient, but which is able to be recognized by the Treg cells of the pharmaceutical composition of the invention.

In a first embodiment, these cells originate from the histocompatible donor and “naturally” (coded in their genetic heritage) carry this antigen, which is absent in the recipient.

Alternatively, there may be autologous cells originating from the recipient (who therefore also constitutes the donor), in which at least one AgMH antigen has been introduced. Preferably, this is carried out by gene transfer using tools known to a person skilled in the art, in particular viral vectors.

In practice, the host receiving such a composition will acquire an immune tolerance to the antigen specifically recognized by the Tregs according to the invention, and will therefore also develop a state of tolerance towards all the antigens carried by these stem cells, initially recognized by the specific regulatory T cells.

In the particular case of male/female transfer, this may involve cells originating from a male donor and therefore carrying male minor antigens such as DBY, UTY and SMCY, for example male bone marrow cells (BM).

Alternatively, they may be bone marrow cells originating from the female recipient, transduced by vectors carrying genes coding DBY and/or UTY and/or SMCY. The use of autologous cells has an obvious advantage in terms of compatibility.

In general and advantageously, they are haematopoietic stem cells (HSC) which ensure the long-term maintenance of the state of tolerance, without myeloablative conditioning nor immuno-suppressor in the recipient. These HSC of medullar origin or derived from various populations of non-embryonic progenitor cells may also possess direct therapeutic power either because of their ability to maintain the state of specific tolerance to one or more AgMH, or by permitting the production of metabolic factors permitting the correction of disorders of genetic origin.

In a first embodiment, they are bone marrow (BM) cells containing haematopoietic stem cells (HSC). These pluripotent cells are destined to give birth to the various types of blood cells during haematopoiesis. These cells are conventionally extracted from medullar or blood samples and purified according to phenotypical markers (for example CD34), or enriched according to functional criteria, such as their capacity to exclude the Hoechst marker. In this second embodiment, the stem cells are also haematopoietic, but undergo an initial purification to isolate a fraction enriched with progenitor cells. This method eliminates a large number of cells which do not have the capacity to be implanted in the recipient and allows the effective genetic modification of a limited number of cells.

In the context of the invention, it is also considered to use genetically modified stem cells, carrying a transgene of interest. These transgenes may be provided by means of a vector, preferably viral. Lentiviral vectors are preferred in the context of therapeutic applications involving HSC. The product of these transgenes is also perceived as a minor histocompatibility antigen on the part of the host recipient of a pharmaceutical composition according to the invention. This transgene can therefore be expressed in addition to the antigen specifically recognized by the Treg cells according to the invention, or may itself constitute this antigen.

Characteristically, the composition according to the invention serves to ensure the stable expression of the transgene.

As a transgene of interest, mention can be made for example of any functional gene capable of compensating for a defective gene in the host, for example the dystrophine gene in patients suffering from DMD.

The composition according to the invention may further contain pharmaceutically acceptable adjuvants or vehicles, known to a person skilled in the art.

The quantity of cells in the pharmaceutical composition claimed is advantageously administered at the rate of 10⁴ and 10⁸ cells per kg of the recipient. In the context of the invention, it has been demonstrated that even a small quantity of the Treg cells were sufficient for effective conditioning. The quantity of Treg cells is advantageously equal to about 10⁶ per kg.

The injection via the bloodstream, preferably intravenous, is the privileged mode of administration of the pharmaceutical composition of the invention. It has been found that a single injection was as effective as a succession of repeated (weekly) injections of equivalent quantities of cells.

The presence of the two cell types in the pharmaceutical composition claimed serves to introduce them simultaneously, during a single injection, into the histocompatible host recipient. However, the advantageous effect on the immune tolerance (useful in gene and cell therapy, for transplantations and grafts) of such a combination is equally well preserved during the simultaneous, separate or time-spread administration of the two cell types described above, which are the subject matter of the composition of the invention.

Another aspect of the invention relates to the use of such a composition for preparing a medicament for increasing or improving the immune tolerance in a histocompatible host.

For recall, a histocompatible host is the recipient of a graft originating from a donor having compatible major antigens but divergent minor antigens, liable to trigger an immune reaction culminating in the rejection of the graft and/or the therapeutic transgene by the host.

In the context of the present invention, several very important observations for therapeutic prospects have been made after conditioning with the two cell types as described:
the immune tolerance of the recipient is improved in the short term and the long term;
the tolerance remains specific because the recipient remains perfectly immunocompetent;
tolerance is established toward the specific minor antigen recognized by the Treg cells, but also towards other antigens of the same type, including towards a transgene initially associated in the graft whereof the stable expression is ensured;
the tolerance persists towards a secondary transplant carrying minor antigens, even if the initial antigen specifically recognized by the Treg cells is not present in the secondary transplant.

In practice, these advantages offer various therapeutic applications concerning transplantations of genetically modified autologous tissues, the gene transfer procedures, and the tolerance induction procedures specific for defined antigens for attenuating the autoreactive reactions in autoimmune pathologies having identified molecular and cell targets.

Firstly, the ability to increase the immune tolerance of the recipient allows for greater flexibility in the choice of the donor, particularly by removing the barrier of the sex difference.

In the case of a transplantation of liquid or solid tissues, for example that of the skin, the grafting of the stem cells is only one preliminary conditioning step, preparatory to the secondary transplant of the tissue of interest, originating from the donor.

The invention therefore also relates to the use of the composition described in the case in which the immune tolerance is increased for the transplantation of a tissue originating from a donor carrying a minor histocompatibility antigen, for example for a skin graft.

The stem cells may be the vector of one or more genes called repair genes, which serve to correct a defect of genetic origin present in the host. The repair gene may be provided by the genetic heritage of the stem cells themselves or introduced into these cells by means of a vector, preferably viral. In this case, it involves a transgene carried by the stem cells.

As previously described, the conditioning described is satisfactory for the claimed therapeutic applications. The medicament according to the invention therefore constitutes a sufficient treatment as such.

The invention and the advantages thereof will appear more clearly from the exemplary embodiments below in conjunction with the appended figures, which are not limiting.

Considering that the engraftment of a male tissue transplant in female recipients generates a clearly defined immune response against the DBY, UTY and SMCY antigens in the C57Bl/6 (B6) mouse (11), the following exemplary embodiment is based on this model to test whether the male-specific Tregs can promote the long term engraftment of the transplant of spinal cord progenitor cells. This model mimics situations in which autologous stem cells, transduced with foreign transgenes, are rejected due to the host's specific responses to the transgene. Moreover, the presence of several male antigens introduces a level of appreciable complexity, which serves to test the extent to which the Tregs directed against a single epitope can inhibit the immune responses induced against all the other proteins expressed. Tregs directed against the DBY male antigen, obtained from Marylin mice carrying a transgenic TCR, has been used as the sole conditioning for obtaining a long term engraftment of appreciable doses of male BM in female B6 hosts. Importantly for therapeutic applications, the B6 mice tolerized by this procedure become permissive towards secondary engraftments of modified autologous tissues with the transgene present in the initial BM, independently of the DBY antigen recognized by the perfused Tregs.

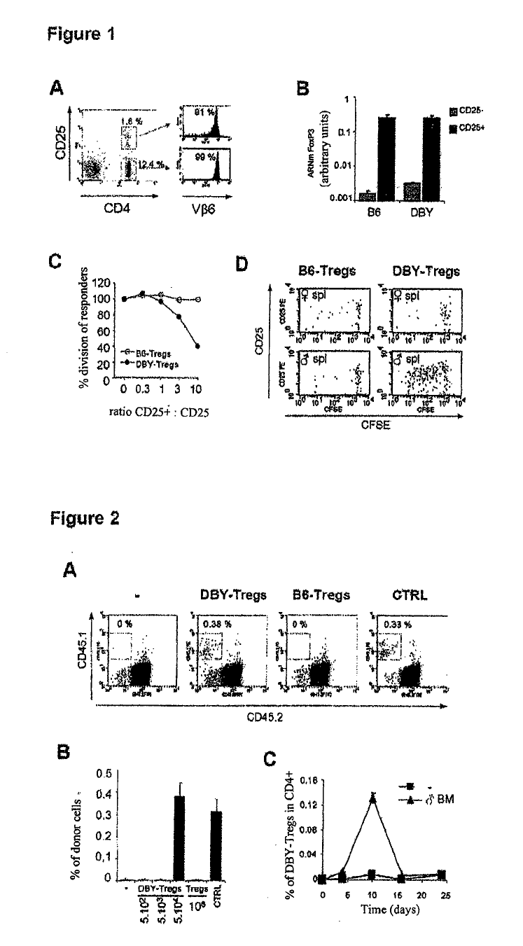

FIG. 1: Expansion in vivo and immuno-suppressive properties of DBY-Tregs.

(A) Analysis by FACS of the cell of the lymph glands of a female Marylin mouse labelled with anti-CD4-FITC, Vβ6-PE and CD25-biotin/APC-streptavidin. The dead cells were excluded with the help of D actinomycin (7-AAD).

(B) The mRNA FoxP3 levels of the CD25+ and CD25− cells purified from female B6 and Marylin mice were determined by real time PCR analysis on fresh splenocytes.

(C) Suppression in vitro by DBY-Tregs, estimated by the percentage division of 5.10³ CD4+CD25− T-helper cells from a Marylin mouse (marked with 2 μM of CFSE), stimulated with 5.10⁵ male B6 splenocytes in the presence of variable doses of DBY-Tregs (solid circles) or non-specific Tregs-B6 (empty circles).

(D) Expansion in vivo of DBY-Tregs: DBY-Tregs marked with 5 μM of CFSE were transferred jointly with 10.10⁶ female or male splenocytes into a CD45.1 recipient female mouse. On day 6, the splenocytes were marked with CD4-Cy, CD25-PE and CD45.2-biotin/APC-streptavidin. The point histograms are windowed on the CD4+CD45.2+ cells.

FIG. 2: Short term male BM engraftment and transient expansion of DBY-Tregs.

(A, B) Short-term engraftment (day 28) of CD45.1 congenic male BM cells (5.10⁶ cells) transferred to female B6 mice, either untreated (−) or conditioned with an intravenous injection of variable quantities of DBY-Tregs or 1.10⁶ B6-Tregs. Male B6 mice were grafted, as positive controls (CTRL). The percentages of CD45.1+ donor cells were analyzed in the PBMC. The results represent the mean of 3-6 mice per group±standard error of mean (SEM).

(C) Transient expression of DBY-Tregs. DBY-Tregs (1.10⁵ cells) were transferred to a female B6 mouse with or without male BM (10⁷ cells). At each time step, two mice were sacrificed and their splenocytes stained with CD45.1-PE, APC-CD4 and 7-AAD. The graph shows the percentage of CD45.1+ cells in the CD4+ 7-AAD-cells. The results represent the mean of 2 mice per group±SEM.

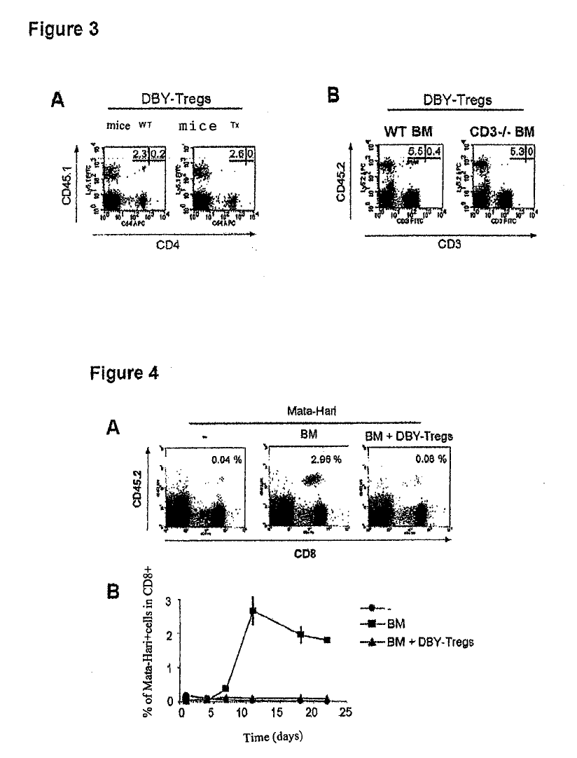

FIG. 3: Tolerance to male antigens mainly takes place via peripheral mechanisms.

(A) Male BM of congenic female CD45.1 B6 mice (15.10⁶ cells) were transferred to intact (n=5) or thymectomized (n=5) B6 mice, conditioned with a single intravenous injection of 1.10⁵ DBY-Tregs.

(B) Male BM of wild B6 mice or CD3−/− mice (15.10⁶ cells) were transferred to congenic female CD45.1 B6 mice (n=5 for each group), conditioned with a single intravenous injection of 1.10⁵ DBY-Tregs.

In (A) and (B), the donor chimerism expressed in % of CD45.1+ cells was analyzed in the PBMC at different time intervals after BM transfer. The FACS colorations shown (day 60) are representative of two experiments.

FIG. 4: Mixed male-female chimerism alters the T cells specific for male antigens.

10⁵ cells of T Mata-Hari CD8 were transferred to congenic CD45.1 female mice either alone (−), or with 10.10⁶ male BM or with 10.10⁶ male BM and 105 DBY-Tregs.

(A) Representative blood samples stained with CD45.2-biotin/APC-streptavidin, CD8-PE and 7-AAD are shown (day 11 after transfer).

(B) The percentage of Mata-Hari cells among the total CD8+ analyzed in the PBMC at different time intervals is shown. The results represent the mean of 2 mice per group±SEM.

FIG. 5: Development of a mixed long-term chimerism and multi-lineage.

(A) Male B6 BM cells (15.10⁶ cells) were transferred to congenic female B6 CD45.1 mice, conditioned with either 5 weekly intravenous injections of 2-5.10⁵ DBY-Tregs (solid circles), or a single injection of 1.10⁵ DBY-Tregs (empty circles). The donor chimerism expressed in % of CD45.2+ cells was analyzed in the PBMC at different time intervals after BM transfer. The results represent the mean of 5 mice per group±SEM.

(B, C) Mice chimerized for more than 300 days (5 injections of DBY-Tregs) were sacrificed and the cells of various organs were analyzed by FACS. The splenocytes were stained with CD45.2-biotin/APC-streptavidin, PE combined with anti-CD8, CD4, B220, CD11c and 7-AAD (B). Thymocytes (C) were stained with CD4-FITC, CD3-PE, CD45.2-biotin/PECy7-streptavidin and CD8-APC (no window) or with CD3-FITC, CD45.2-biotin/PE-streptavidin, 7-AAD and CD11c-APC (FACS windowed on CD3-7-AAD is shown).

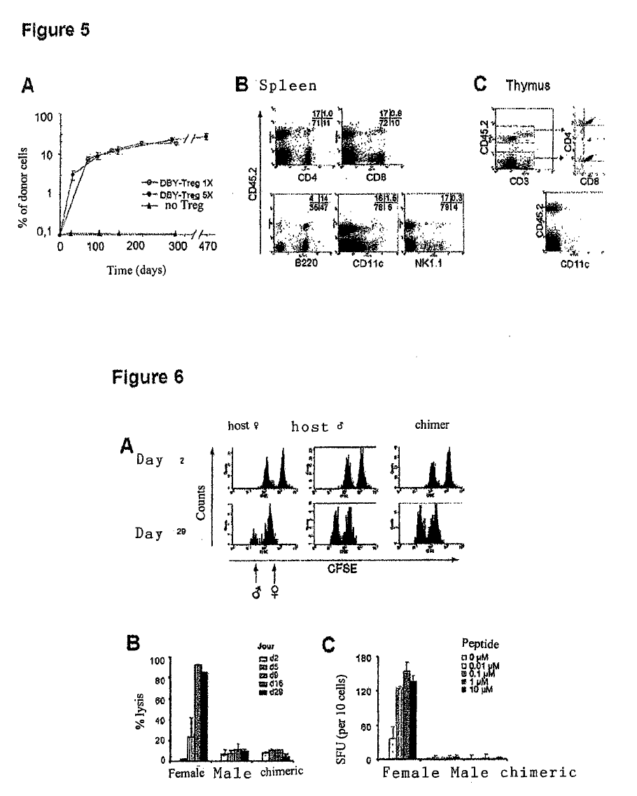

FIG. 6: High tolerance to male antigens in chimeric mice.

(A, B) Absence of anti-male CTL activity in the chimeric mice. The male and female splenocytes marked with 0.5 μM and 5 μM of CFSE, respectively, were transferred to female mice, male mice or chimeric female mice. The PBMC were marked with PE anti-B220 and 7-AAD at various time intervals and the percentages of specific lyses of the male on female splenocytes were calculated, as shown in detail in the Material and Methods section. The results represent the mean of two mice per group±SEM.

(C) Absence of anti-male response by the T cells. Female, male or chimeric mice were subcutaneously provoked with 50 μg of UTY peptide emulsified in IFA. The splenocytes were tested on day 10 in a standard IFNγ ELISPOT test against various doses of UTY peptide. The results represent the mean of 3 mice per group±SEM.

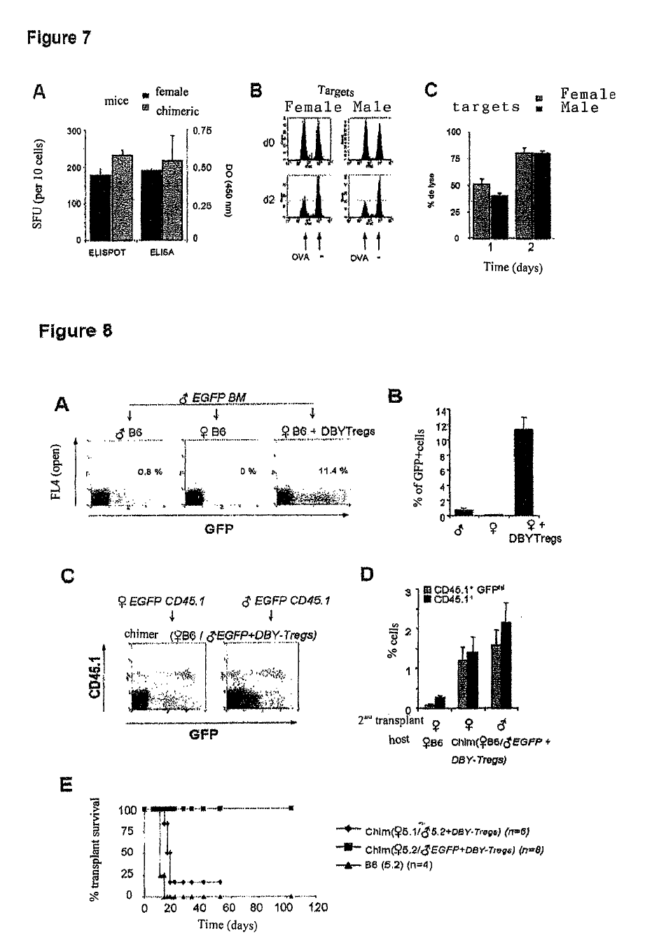

FIG. 7: Haematopoietic chimerism does not affect the immune response to third party antigens.

(A) Chimeric or naive female mice were provoked subcutaneously with 100 μg of OVA protein emulsified in IFA. The splenocytes were tested on day 10 in a standard IFNγ ELISPOT test against the OVA257 epitope. The serums were tested by ELISA for the anti-OVA antibodies. The results represent the mean of 3 mice per group±SEM.

(B, C) Susceptibility of male cells to cytolytic immune activity. Chimeric mice were provoked subcutaneously with OVA protein in IFA as in (A), and were perfused on day 8 with male splenocytes (black bars) or female splenocytes (grey bars), pulsed with OVA257 (0.5 μM of CFSE) or not pulsed (5 μM of CFSE). The PBMC were analyzed on days 0, 1 and 2. The results represent the mean of 2 mice per group±SEM. The percentage lyses specific of pulsed cells on non-pulsed cells was calculated as previously (FIG. 6) with male on female cells.

FIG. 8: Engraftment of secondary tissue graft expressing the EGFP transgene in the absence of DBY antigen.

(A) 7.10⁶ male BM cells of transgenic EGFP mice were transferred to male B6 hosts, female B6 hosts, or female B6 hosts conditioned with 10⁵ DBY-Tregs. The FACS analysis representative of 3 experiments is shown (day 150).

(B) Donor chimerism expressed in % of EGFP+ cells analysed in PBMC 5 months after the transfer of EGFP BM according to (A). The results represent the mean of 3 mice per group±SEM.

(C, D) BM cells from male or female EGFP×CD45.1 mice were transferred to chimeric or naive female EGFP mice. The reading representative of FACS (C) and the percentage of EGFP^{high} and CD45.1⁺ (D) cells in the PBMC after 5 months are shown. The results represent the mean of 3 mice per group±SEM.

(E) Engraftment of long-term graft of transplantations of female EGFP skin in chiremic male/female EGFP mice (n=8, solid squares). In the controls, 5/6 of the chimeric CD45.2/CD45.1 mice devoid of EGFP (solid squares) and 4/4 of female B6 mice (solid triangles) rejected the female EGFP skin graft between day 12 and day 16. The results presented are derived from two independent experiments.

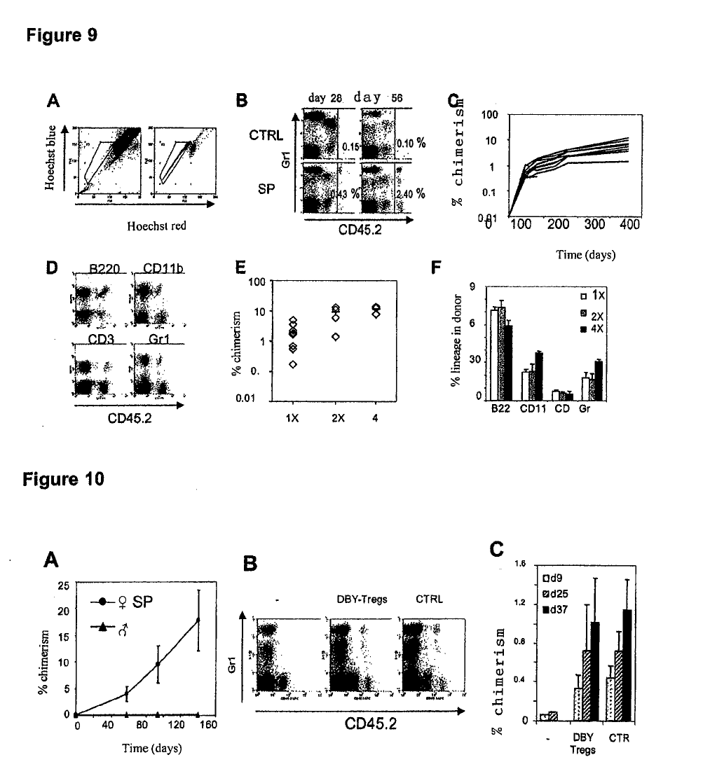

FIG. 9: Transplantation of purified haematopoietic stem cells without host preconditioning.

(A) Analysis by FACS of the SP profile of bone marrow cells, in the absence or presence of verapamil. (B-D) Development of a haematopoietic chimerism over time. Recipient female CD45.1 mice were injected intravenously 4 days in succession with PBS (CTRL) or with 10⁵ SP cells from B6 mice (SP). The percentage of donor cells was analyzed in the PBMC by FACS. Representative data on day 28 and day 56 (B); observation over time mouse by mouse, group SP (C) and multi-lineage transplantation, group SP (D). (E-F) Recipient female CD45.1 mice were injected intravenously with 105 SP cells from B6 mice 4 days in succession (4×), 2 days in succession (2×) or only on day 0 (1×). Percentage of chimerism in the PBMC (E) and multi-lineage reconstruction (F) are indicated. The results represent the mean of 3-8 mice per group±the standard error of mean (SEM).

FIG. 10: Haematopoietic stem cells unpaired on minor antigens can transplant in the presence of specific Tregs.

(A) Rejection of male HSC by female mice. Recipient female CD45.1 mice were injected intravenously on day 0 with 10⁵ SP cells from female or male B6 mice. The percentage of donor cells was analyzed in the PBMC by FACS over time. The results represent the mean of 3 mice per group±standard error of mean (SEM). (B-C) Rejection is inhibited by Tregs. Recipient female CD45.1 mice were injected intravenously on day 0 with 10⁵ SP cells from male B6 mice in the absence (NO) or presence of 3.10⁵ DBY-Tregs. In the control group (CTRL), the recipient male CD45.1 mice were injected with SP from male B6 mice. The percentage of donor cells was analyzed in the PBMC by FACS over time. The results represent the mean of 3 mice per group±the standard error of mean (SEM).

### MATERIAL AND METHODS

### Mice

Mice aged from 6 to 8 weeks C57BI/6 (CD45.2) and congenic Ly5.1 mice (PtprcaPep3b/BoyJ [CD45.1]) were obtained from Charles River (L'Arbresle, France). Marylin mice carrying a recipient with transgenic T cells specific for the DBY peptide (NAGFNSNRANSSRSS) (SEQ ID 1), complexed with JAb (18) were donated by O. Lantz and the females were used here on a genetic B6 RAG2+/− background (CDTA, Orleans, France). Mata-Hari mice RAG1 −/− carrying a transgenic TCR specific for the UTY peptide (WMHHNMDLI) (SEQ ID 2) complexed with D^{b} (21) were also donated by O. Lantz. Transgenic hemizygotic EGFP mice (C57BL/6-Tg(ACTB-EGFP) 1Osb/J, Jackson Laboratory, USA) express the cDNA of EGFP under the control of a chicken beta-actin promoter and the cytomegalovirus amplifier. EGFP mice were interbred with CD45.1 mice to generate EGFP×CD45.1 mice. The two species were raised in our animal installations. All the animal experiments were conducted according to the institutional directives for the care and use of animals.

Analysis by FACS (Fluorescence-Activated Cell Sorting)

All the reagents were obtained from BD-PharMingen (Le Pont de Claix, France). The erythrocytes were removed by hypotonic shock with PharMLysis buffer. The peripheral blood mononuclear cells (PBMC) were incubated for 10 minutes at 4° C. with 2.4G2 antibodies against the FcII/III recipients, and then stained for 30 minutes in PBS—0.1% bovine serum-albumin with saturating quantities of combinations of the following mABs: anti-CD3, anti-CD4, anti-CD11c, anti-B220 and anti-CD45.1 combined with fluorescein isothiocyanate (FITC), anti-CD8, anti-CD11b, anti-CD25, anti-Gr1, anti-CD45.2, anti-NK1.1 and anti-Vβ6 combined with phycoerythrine (PE), anti-CD45.2 combined with biotin and streptavidin combined with allophycocyanine (APC). The dead cells were excluded by using a staining with 7-actinomycine D (Sigma Chemical Co, St. Louis, Mo.). The flux cytometry analysis was performed on a FACSCalibur, using the CELLQuest software (BD).

Purification of Regulatory T Cells

The splenocytes and cells of the lymph glands were incubated with saturating quantities of biotinylated (7D4) anti-CD25 and microspheres of streptavidin (Miltenyi Biotec, Paris, France), followed by magnetic separation of the cells using LS columns (Miltenyi Biotec), according to the manufacturer's instructions. The cells were then stained for 30 minutes on ice with streptavidin-FITC, sorted on a MoFlow (DakoCytomation, Freiburg, Germany) and injected into the tail vein, in 0.2 ml of PBS.

Analysis of In Vitro Inhibition

Purified CD45.2 CD4+CD25− T cells, from Marylin mice, were marked with CFSE (Molecular Probes, Cambridge, United Kingdom). In short, 2×10⁷ cells/mL were incubated with 2 μM of CFSE at 37° C. for 10 minutes in RPMI 1640, and then washed twice. 5.10³ cells were then cultivated in U-bottom plates with 5.10⁵ male splenocytes of congenic CD45.1 mice and various proportions of CD4+CD25+ Tregs from B6 or Marylin mice. On day 3, the cells were stained with CD4-PE, 7-AAD and CD45.2-biotin/streptavidin-APC before analysis by FACS. The division (in percent) of the responding CD25− cells was determined as the percentage of cells having undergone at least two divisions with regard to the total quantity of responding cells which were divided in the absence of Tregs.

Bone Marrow Transplantation

The donor bone marrow was extracted with PBS from femurs and tibias. The erythrocytes were lysed with ACK buffer and the bone marrow cells (BM) were injected into the tail vein in 0.2 mL of PBS 1× in the presence or absence of CD4+CD25+ Tregs.

Analysis of In Vivo Mortality

Splenic female and male C57BI/6 cells, or splenic sex-assorted cells carrying or not carrying 10 μM OVA257 (2×10⁷/mL in RPMI 1640) were incubated with 5 μM or 0.5 μM CFSE (Molecular Probes, Cambridge, United Kingdom) at 37° C. for 10 minutes. After washing, the cells were mixed, and 2×10⁷ cells were injected into the tail vein in 0.2 mL PBS 1×. The PBMC were collected from individual mice at regular time intervals, marked with PE anti-B220 and 7-AAD and analyzed for CFSE expression by FACS. The percentage of lyses specific for the male cells with regard to the female cells (m/f) was calculated on the B220+ cells at each time interval tₓ as follows: % specific lyses=[(m/f)t₀−(m/f)tₓ]/[(m/f)t₀]×100. The quantities m and f were measured on standard gates placed on the peaks of the low male and high female CFSE histograms.

Anti-Ovalbumin Immune Responses

The mice were subjected to subcutaneous provocation at the base of the tail with 100 μg of ovalbumin protein (Sigma) or 50 μg of OVA257 (SIINFEKL) peptide (Epytop, Nîmes, France), emulsified in an incomplete Freund's adjuvant (Difco laboratories, BD). Analyses by IFNγ-ELISPOT and ELISA were performed 8 to 10 days later, as already described (17). In short, for the IFNγ-ELISPOT analysis, freshly isolated splenocytes (2×10⁶/well and dilutions in series) were cultivated in complete medium with or without 10 μM of OVA257. For each test, Con A was added (5 μg/ml) as positive control. After 20 h, the spots were developed and counted using a Bioreader 2000 (BIO-SYS, Karben, Germany). The spot formation units (SFU) are shown after subtraction of the background noise obtained with non-pulsed splenocytes.

Analysis by PCR in Real Time

The total RNA were extracted using the RNAeasy Micro Kit (QIAGEN) with 10⁵ isolated cells of each population. The cDNA was synthesized from each batch of RNA using reverse transcriptase MuLV and random hexamers as initiators (Applied Biosystems). The real time PCR was carried out on an ABI prism 7700 using Absolute QPCR ROX Mix (ABgene) in duplicate and average threshold cycles (Ct) of the duplicates were used to calculate the level of mRNA Foxp3 in each population. All the mRNA levels reported were normalized to the mRNA mPO level, where mPO=1. The PCR initiators were as follows:

| | |
|---|---|
| Foxp3: | |
| 5′-GGCCCTTCTCCAGGACAGA-3′ | (SEQ ID 3) |
| 5′-GCTGATCATGGCTGGGTTGT-3′ | (SEQ ID 4) |
| 5′-ACTTCATGCATCAGTCTCCACTGTGGAT-3′ | (SEQ ID 5) |
| mPO: | |
| 5′-CTCCAAGCAGATGCAGCAGA-3′ | (SEQ ID 6) |
| 5′-ATAGCCTTGCGCATCATGGT-3′ | (SEQ ID 7) |
| 5′CCGTGGTGCTGATGGGCAAGAA-3′. | (SEQ ID 8) |

Skin Graft

Skin grafts 1 cm in diameter were prepared from backs of female EGFP mice and were transplanted on flanks of the recipients, using a tissue adhesive (3M Vetbond, France) instead of a surgical suture. The bandages were removed on day 5. The grafts were observed every 2 to 3 days up to day 20, and then each week, and recorded as rejected when less than 10% of viable tissue persisted.

Purification of HSC

Marrow cells were prepared from femurs and tibias. After lyses of the erythrocytes by hypotonic shock, the cells were resuspended in 10⁶/ml in PBS containing 1% autologous serum and marked with 6.5 μg/ml of Hoechst 33342 (Sigma) for 90 minutes at 37° C. The controls were incubated in the presence of 100 μM of verapamil. The cells were then washed and marked with 7-actinomycin D (7-AAD, Sigma Chemical Co, St., MO). The sorting and analysis of the cells excluding the Hoechst (SP) was carried out on a MoFlow (DakoCytomation, Freiburg, Germany). The SP cells generally accounted for 0.04% to 0.05% of the total marrow cells.

Results

Antigen-Specific Activity of CD4+CD25+ Treg Cells of Marylin Mice

In the B6 strain, the female mice reject the H2-paired male BM transplants, generating CD4 T cell responses against a DBY peptide in the context of the I-A^{b} molecules and dependent CD8 helper responses against the restricted epitopes UTY and SMCY (11). As sources of Treg directed against a given male antigen, CD4+CD25+ Tregs were isolated from Marylin mice expressing a transgenic TCR, specific for the restricted male epitope I-A^{b} DBY NAGFNSNRANSSRSS (SEQ ID 1) (18).

Phenotypically, the female RAG+/− Marylin mice have a distinct population of CD4+CD25+ (DBY-Tregs) T cells expressing the Vβ6 TCR transgene and representing 6-11% of the total CD4+ T cells (FIG. 1A). The phenotype of these regulatory T cells was confirmed by quantifying the lineage/differentiation marker for the recently identified Tregs, FoxP3 (FIG. 1B). As detected by PCR in real time, the purified CD25+ from Marylin mice express similar levels of FoxP3 to those observed in the B6-Tregs (B6-Tregs) and 80 times higher than those found in CD25− T cells. The specificity and the suppressive function of the DBY-Treg was characterized in vitro and the inhibition of the proliferation of the CD4+CD25− naïve Marylin T cells (DBY-T-helper), stimulated with male splenocytes and marked with CFSE, was observed. As shown in FIG. 1C, DBY-Tregs inhibit the proliferation of the DBY-T-helper in a dose-dependent manner, whereas the non-specific B6-Tregs are ineffective in any ratio.

The antigenic specificity of the DBY-Tregs was then verified in vivo by their proliferation capacity in female recipients provoked with female or male splenocytes. As measured with the CFSE dilution experiments, a vigorous expansion of the DBY-Tregs was observed on day 6 (FIG. 1D). This only occurred after the immunisation with male splenocytes. The B6-Tregs essentially remained undivided in all conditions (FIG. 1D). In conclusion, the DBY-Tregs of Marylin mice proved to be effective in vitro suppression experiments and were activated in a male-specific manner in vivo.

Induction of Mixed Haematopoietic Chimerism Using DBY-Tregs

The capacity of these mono-specific Tregs to induce mixed chimerism, without preconditioning, was investigated. DBY-Tregs cells were used to promote the direct engraftment of a transplant of a moderate dose of male BM in female B6 hosts, in the absence of myeloablation and immuno-suppression. As control in the absence of immune responses, the injection of 8.10⁶ CD45.1+ congenic male BM gave rise to a chimerism of 0.3-0.4% in male CD45.2+ mice (FIG. 2A). The same transplant was completely rejected in female CD45.2+ mice on day 28. In these female mice, the engraftment of the transplant was already compromised on day 14 and virtually absent on day 21 (results not shown). As expected, the perfusion in females of DBY-Tregs, concomitantly with the male BM, suppressed the rejection of the transplant and served to achieve nearly 0.4% of chimerism (FIG. 2A). The dose of DBY-Tregs was then adjusted and an on/off effect was observed on the engraftment of the BM transplant between 5.10⁴ and 5.10³ DBY-Tregs (FIG. 2B). Significantly, the doses of DBY-Tregs above the threshold promoted a maximum engraftment in all the female mice, achieving an identical level to that in the control male recipients, treated with the same quantity of BM cells. As for the in vitro suppression, a higher dose of non-specific 10⁶ B6-Tregs was ineffective (FIG. 2B). In conclusion, these results emphasized the extreme potential of the CD4+CD25+ T cells specific for DBY in promoting mixed haematopoietic chimerism.

Given the extremely small number of DBY-Tregs required, the question of their proliferation parallel to the engraftment of the male BM transplant can be asked. In fact, an expansion of 10-15 times the DBY-Tregs was observed in the spleen on day 10 (FIG. 2C). This agrees with the high proliferative capacity observed against the male splenocytes in vivo (FIG. 1D). Advantageously, the DBY-Tregs fall back to lower levels on day 16 without compromising the long term BM engraftment on day 28 (FIG. 2B), suggesting that other mechanisms than active suppression by DBY-Tregs are involved later.

Peripheral Mechanisms are Sufficient to Induce Mixed Chimerism

Transplantation in an allogenic context often requires conditioning that leads to the generation by the thymus of a partially or totally novel repertory of T cells, as the main cause of the induction of tolerance. In the system according to the invention, without myeloablation or immuno-suppression, the mature peripheral T cells of the host are the first barrier to be crossed. To examine whether induction of tolerance initiated by DBY-Tregs only involves peripheral mechanisms, male BM and DBY-Tregs were transferred together either into wild mice, or into thymectomized mice. It turned out that the two groups demonstrate a comparable chimerism in all the mice at two months (FIG. 3A), demonstrating that peripheral mechanisms are sufficient to promote chimerism in mice treated with DBY-Tregs.

To determine whether the donor bone marrow requires mature donor lymphocytes to induce tolerance, male CD3−/− BM, devoid both of T CD25− and CD25+ lymphocytes, were transplanted in female CD45.1 mice conditioned with DBY-Tregs. As shown in FIG. 3B, the absence of mature donor T cells has no effect on the engraftment of the BM transplant.

Coupled with the transient expansion of DBY-Tregs, these results encouraged an examination of the future of anti-male CD8+ T cells after a BM transfer and the perfusion of Tregs. For this purpose, the Mata-Hari mouse model expressing a transgenic TCR, specific for the restricted UTY D^{d} peptide WMHHNMDLI (SEQ ID 2) (21) was used. The Mata-Hari CD8+ T cells were transferred and traced in congenic female CD45.1 mice, alone, perfused with male BM, or perfused with male BM plus DBY-Tregs. On day 11 after the transfer of BM, the proportion of Mata-Hari CD8+ T cells increased dramatically, compared to the non-stimulated cells, reaching 2.6±0.4% of CD8+ T cells on day 11 (FIG. 4A-B). In contrast, in the presence of DBY-Tregs, the Mata-Hari cells only accounted for 0.1±0.03% of the CD8+ T cells. Even later, when the DBY-Tregs were difficult to detect (FIG. 2C), the Mata-Hari never exceeded 0.1±0.08% of the total CD8+ T cells on day 22 (FIG. 4B). These results demonstrate that the proliferation and the function of the CD8+ T cells specific for male antigens are altered at the start of the induction of the mixed chimerism.

Development of a Long-Term Mixed Chimerism and Multi-Lineage

DBY-Tregs effectively inhibited the anti-male immune response promoting the short-term engraftment of the BM transplant, but their transient expansion could be associated exclusively with a short-term suppression, compromising the longer term engraftment of the HSC transplant and the donor-specific tolerance.

The action of a single injection of 10⁵ DBY-Tregs corresponding to twice the threshold dose determined in FIG. 2B, was compared with 5 weekly injections of 2-5.10⁵ DBY-Tregs, on the development of a long-term chimerism (FIG. 5A). The two protocols maintain sustained and high levels of donor chimersim in the peripheral blood lymphocytes between 3 and 4 months (9.1±1.8 and 12.3±1.0 respectively), with a gradual increase at 9 months (21.8±2.6 and 18.0±1.7 respectively) and a systematic longer term engraftment at 18 months for all the mice. No BM engraftment took place with high doses of 10⁶ non-specific B6-Tregs, even preactivated with anti-CD3 antibodies (results not shown). The chimerism was obtained in all the haematopoietic compartments (CD4+, CD8+, B220+, NK1.1+ and CD11c+) in 100% of the recipients, even in those conditioned with a single low dose of 10⁵ DBY-Tregs (FIG. 5B). Significantly for the maintenance of the long-term chimerism, the spinal cord was also colonised by donor HSC, as defined by their Lin-Sca+c-Kit phenotype or the Hoechst exclusion of the lateral population (results not shown). Dendritic CD11c+ donor cells were also present in the thymus, as well as normally developing thymocytes, as demonstrated by the CD4+, CD8+ and CD4+CD8+ percentages (FIG. 5C). This result suggests that the repertory of T cells generated in the recipient thymus may be educated by the haematopoietic cells of both recipient and donor origin.

Robust Tolerance to Male Antigens and Immunocompetence to Third Party Antigens

By creating a multi-lineage long-term chimerism, a robust tolerance was hoped for towards all the antigens present in the transplant. To confirm this, mice displaying a minimum of 3-5% of chimerism were provoked with various formulations of male antigen. Cytotoxic analyses in vivo were performed, by perfusing 5.10⁶ male or female B6 splenocytes, marked with two levels of CFSE. In this system, naïve female mice were sensitized and reacted selectively against male splenocytes on day 10 after the perfusion, eliminating virtually all the male cells on day 16, whereas no cytotoxicity had been observed in the male hosts (FIG. 6A). As expected, the chimerized mice demonstrated no rejection of the male targets during the 29 days of the experiment (FIG. 6B), indicating that the long-term peripheral tolerance to male antigens had been achieved.

This tolerance could be due to an active and sustained regulation process and/or an alteration/suppression of the anti-male T cells. A sustained suppression by the DBY-Tregs appeared unlikely since they were difficult to detect, being below the FACS detection threshold in the blood and in all the primary or secondary lymphoid organs tested several months after BM transfer (results not shown). To clarify this point, the responses to the CD8 and CD4 epitopes were uncoupled to avoid a potential immune suppression by the residual DBY-Tregs. Thus, mice were provoked with the well characterized UTY epitope in the presence of the strong IFA adjuvant. No anti-male response was detected, as demonstrated by the absence of a peptide specific IFNγ response in an ELISPOT test (FIG. 6C).

To eliminate the possibility of a general state of non-response in the chimera, the immune response to a third party antigen, absent from the initial transplant, was examined. After immunization with OVA, the frequency of the antigen-specific IFNγ producing T cells and the assays of the OVA-specific IgG antibodies were monitored. The chimeric mice and controls responded equally to this antigen (FIG. 7A). It was determined whether the specific tolerance of the male antigens affected the rejection of the male splenocytes loaded with the OVA257 peptide. By using the in vivo cytotoxicity experiments, it was found that the male splenocytes carrying the peptide were just as susceptible to the CTL lyses as the female cells carrying the peptide (FIG. 7B, C). Significantly, despite a high cytotoxicity against male cells carrying third party antigens, the long-term chimerism was unaffected (results not shown).

In conclusion, a robust tolerance towards all the antigens initially present in the transplant, such as UTY protein, was observed, but not towards the third party antigens introduced later. Moreover, this tolerance does not affect the normal immunity directed against the donor target cells.

Applications to the Secondary Engraftment of Engineered Tissues

The absence of immune responses observed here towards male antigens in chimeric mice is similar to the long-term transgene-specific tolerance necessary for completely safe gene transfer applications. It was therefore decided to extend this robust tolerance to foreign transgenes by developing a corresponding molecular chimerism in addition to the male protein. It was decided to introduce the EGFP protein (Enhanced Green Fluorescent Protein), and, as sources of modified BM to express the EGFP protein (ACTB-EGFP), transgenic male C57BL/6-TgN mice. As previously demonstrated with skin graft experiments (8), EGFP and certainly other associated minor antigens precipitate the rejection of the male EGFP^{high} BM cells in the male hosts (FIG. 8A). As expected, the rejection of all the male EGFP BM cells took place in the untreated females, probably due to a strong anti-male response (FIG. 8A). On the contrary, females conditioned with 10⁵ DBY-Tregs demonstrated a stable chimerism, up to 10% at 5 months, with a strong expression of the EGFP transgene (FIG. 8A, B). These results demonstrate that a single perfusion of DBY-Tregs directed against a single MHC epitope in class II of DBY is sufficient to induce a long-term tolerance to all the other minor antigens present in the transplant.

Finally, it was determined whether the strong tolerance developed towards all the donor antigens present in the first transplant could be exerted in the absence of an expression of DBY. For this purpose, female B6 mice chimerized with a male EGFP (CD45.2+) bone marrow and DBY-Tregs for 5 months (FIG. 8A, B) were transplanted a second time with male or female EGFP BM cells, carrying a traceable CD45.1 marker. It was found that the CD45.1+EGFP^{high} cells of male or female origin where grafted equivalently, demonstrating that the tolerance applied to all the minor antigens, even in the absence of a coexpression of DBY (FIG. 8C, D).

To extend these results to the stringent context of organ transplantation, skin grafts were used as secondary transplants. Naïve female B6 mice rejected the EGFP skin graft rapidly between days 12 and 16 (FIG. 8E). In contrast, 100% of the female B6 mice chimerized with male EGFP BM cells tolerated their implant, accepting the skin graft indefinitely (n=8). Significantly, control mice chimerized with male CD45.2 BM, which were not tolerized towards EGFP, did not accept this allograft of a third party (5/6) EGFP, illustrating the strength and specificity of the tolerance created towards the EGFP transgene.

Transplantation of Purified Haematopoietic Stem Cells without Preconditioning of the Host

We decided to purify the haematopoietic stem cells on their capacity to exclude the Hoechst. Only 5 of these cells, called a side-population (SP) and of which the phenotype is Lin− ckit+ Thy1.1low Sca-1+ Flk2− CD34− can effectively reconstitute irradiated hosts. Here, the SP cells sorted from four CD45.2 mice, on their capacity to exclude the Hoechst (FIG. 9A), were injected intravenously into congenic CD45.1 mice in the absence of any myeloablative conditioning. Whereas 4 weeks after the transplantation, less than 0.5% of chimerism was observed in the peripheral blood (FIG. 9B), this percentage increased rapidly to reach more than 2% at 6 weeks. It should be noted that this chimerism was maintained for more than one year and in all the recipients (FIG. 9C), and concerned the mono/granulocytic lineages as well as B and T (FIG. 9D). We also varied the dose of the injected SP cells and found that a maximum chimerism could be achieved with 2.10⁴ SP cells, purified from 2 donors (FIG. 9E). Injecting 1.10⁴ SP cells, purified from a single donor marrow, leads to the development of the chimerism in all the mice but with variable engraftment percentages, revealing that empty haematopoietic niches still appear to exist. The multi-lineage transplantation is more or less identical to all these various doses (FIG. 9F). Haematopoietic Stem Cells not Paired on Minor Antigens can Graft in the Presence of Specific Tregs

As expected in the male/female rejection system, male HSC transplanted in female mice are rejected, as opposed to the combination compatible with female HSC (FIG. 10A). In contrast, the simple conditioning of the recipient mice by DBY-Tregs suffices to inhibit this rejection, leads to the same level of engraftment as the control mice and the development of a haematopoietic chimerism over more than one year (FIG. 10B-C). This immuno-suppression is completely specific because the same number of Tregs non-specific for the male antigen are incapable of engendering such an effect, and depends on the regulatory properties of the injected T cells, because CD4+CD25− purified from the same Marylin mouse are ineffective (data not shown). It is thus demonstrated that the results obtained with the bone marrow can be reproduced with purified HSC.

In short, it has been demonstrated that a minimum conditioning with a small number of CD4+CD25+ directed against a single DBY epitope favours the engraftment of BM cells and establishes an antigen-specific tolerance, towards multiple minor histocompatible antigens in haploidentical adults. Advantageously, a secondary transplant carrying one of these minor antigens, such as the EGFP transgene, persists, whether the original antigen having induced the tolerance is present or not. This opens up prospects for the therapeutic application of mixed chimerism for conditioning the host for the subsequent transplantation of tissues or for gene transfers (6). Mono-specific Tregs can now be multiplied in vitro (19) and the de novo generation of Tregs specific for antigens was recently obtained in humans (20). With the advent of such protocols, the mono-specific Tregs could provide new opportunities for replacing the myeloablative immuno-depressive and potentially mortal treatments for the transplantation of BM or autologous HSC genetically modified before the gene transfer.

### REFERENCES

1. Bessis, N. et al. Gene Ther 11, Suppl 1:S10-17. (2004).
2. Jooss, K. et al. Gene Ther 10, 955-963. (2003).
53. Brown, B. D. et al. Blood 100, 1133-1140. (2002).
4. Herzog, R. W. et al. Mol Ther 4, 192-200. (2001).
5. Sarukhan, A. et al. J Virol 75, 269-277. (2001).
6. Sykes, M. Immunity 14, 417-24. (2001).
7. Nikolic, B. et al. Diabetes 53, 376-83. (2004).
8. Andersson, G. et al. Mol Ther 8, 385-91. (2003).
9. Wekerle, T. et al. Nat Med 6, 464-9. (2000).
10. Lin, C. Y. et al. Nat Immunol 3, 1208-13. (2002).
11. Simpson, E. et al. Eur J Immunogenet 28, 505-13 (2001).
12. Waldmann, H. & Cobbold, S. Science 305, 209-12. (2004).
13. Walsh, P. T et al. J Clin Invest 114, 1398-403. (2004).
14. Taylor, P. A. et al. Blood 104, 3804-12. (2004).
15. Trenado, A. et al. J Clin Invest 112, 1688-96. (2003).
16. Joffre, O. et al. Blood 103, 4216-21. (2004).
17. Gross, D. A. et al. Blood 102, 4326-8. (2003).
18. Thery, C. et al. Nat Immunol 3, 1156-62. (2002).
19. Tarbell, K. V. et al. J Exp Med 199, 1467-77. (2004).
20. Walker, M. R. et al. Proc Natl Acad Sci USA (2005).
21. Valujskikh, A. et al. Nat Immunol 3, 844-51. (2002).

1. Pharmaceutical composition comprising:
CD4+ CD25+ regulatory T cells specific for one or more minor histocompatibility antigens; and
stem cells carrying at least one of said antigens.

2. Pharmaceutical composition according to claim 1, wherein the CD4+ CD25+ regulatory T cells are monospecific.

3. Pharmaceutical composition according to claim 1, wherein the CD4+ CD25+ regulatory T cells and/or the stem cells are autologous.

4. Pharmaceutical composition according to claim 1, wherein the minor antigens Comprise a male minor antigen.

5. Pharmaceutical composition according to claim 1, wherein the stem cells are transduced by a vector carrying a gene coding for the minor antigen.

6. Pharmaceutical composition according to claim 1, wherein the stem cells carry at least one transgene.

7. Pharmaceutical composition according to claim 1, as a combination composition for simultaneous, separate or time-spread use in cell or gene therapy.

8.-11. (canceled)

12. Pharmaceutical composition according to claim 1, wherein the stem cells are haematopoietic.

13. Pharmaceutical composition according to claim 4, wherein the male minor antigen is selected from the group consisting of DBY, UTY and SMCY.

14. Pharmaceutical composition according to claim 6, wherein the at least one transgene is introduced by a viral vector.

15. A method for increasing immune tolerance in a histocompatible host, said method comprising administering to an individual in need thereof, a therapeutically effective amount of the pharmaceutical composition of claim 1.

16. The method of claim 15, wherein immune tolerance is increased for stable expression of the transgene.

17. The method of claim 15, wherein tolerance is increased in the host for a tissue transplanted from a donor carrying at least the said antigen.

18. The method of claim 17 wherein the tissue is a skin graft.
